(19) United States
(12) Patent Application Publication
(10) Pub. No.: US 2026/0071173 A1
(43) Pub. Date: Mar. 12, 2026

(54) CONSTRUCTION METHOD OF DOUBLE IMMOBILIZED RECOMBINANT YEAST ENGINEERING BACTERIA, ENGINEERING BACTERIA, CATALYST AND APPLICATION IN SYNTHESIS OF REBAUDIOSIDE

(71) Applicant: Dalian University of Technology, Dalian City (CN)

(72) Inventors: Yimin LI, Dalian City (CN); Wenjie YUAN, Dalian City (CN)

(73) Assignee: Dalian University of Technology, Dalian City, LN (CN)

(21) Appl. No.: 19/390,413

(22) Filed: Nov. 14, 2025

(30) Foreign Application Priority Data
Nov. 5, 2024 (CN) 2024115637590

(51) Int. Cl. C12N 1/20; C07K 14/33; C07K 14/39; C12N 1/18; C12N 9/10; C12N 11/00; C12N 15/63; C12P 19/56; C12R 1/84; C12R 1/865

(52) U.S. Cl. CPC C12N 1/205; C07K 14/33; C07K 14/39; C12N 1/18; C12N 9/1062; C12N 11/00; C12N 15/63; C12P 19/56; C12Y 204/01013; C12Y 204/01017; C07K 2319/02; C12R 2001/84; C12R 2001/865

(57) ABSTRACT

A construction method of double immobilized recombinant yeast engineering bacteria, engineering bacteria, a catalyst and an application in synthesis of rebaudioside. In the present invention, yeast is used as the chassis cell, an ordered self-assembled multi-enzyme cascade surface display system is constructed, and carrier-free immobilization is coupled with uridine piphosphate glucose (UDPG) in-situ regeneration technology, resulting in a novel yeast whole cell catalyst with double immobilization of enzymes and bacteria. The catalyst uses cheap steviol glycoside (St) and rebaudioside A (Reb A) as substrates, and can synthesize rebaudioside (Reb D/M) in an efficient and economical one-pot method without additional addition of expensive UDPG. The synthesis process does not require cumbersome operations including cell disruption, enzyme separation, purification and immobilization, and avoids bottleneck problems in the related art including material transmembrane transport resistance, mass transfer resistance and product hydrolysis by intracellular enzyme. The catalyst has reusability, and can realize continuous and high-intensity rebaudioside biosynthesis through strain rejuvenation, which provides strong technical support for its industrial application.

### TECHNICAL FIELD

The present invention relates to the technical field of preparation of double immobilized recombinant yeast strains and synthesis of rebaudioside, and in particular to double immobilized recombinant yeast engineering bacteria and an application in synthesis of rebaudioside.

### BACKGROUND

Over the past few decades, to reduce the intake of high-calorie sugars, low-calorie or zero-calorie artificial sweeteners have been developed and commercialized as functional substitutes for sugars, among which acesulfame-K, sucralose, and aspartame are the most widely used. Although artificial sweeteners meet the human demand for sweetness, the artificial sweeteners are often reported to have potential health risks (disrupting the balance of human intestinal flora and Class 2B carcinogens, etc.), which are questioned by consumers, and their market share is declining year by year. Therefore, natural sweeteners with high safety, low calories and high sweetness have become the most popular new sugar substitutes among consumers.

Steviol glycosides (SGs), which are derived from natural extracts from stevia rebaudiana leaves, are called “the three major sugar sources in the world” together with sugarcane sugar and beet sugar. SGs are recognized as Generally Recognized As Safe (GRAS). As food additives, SGs have been recognized by organizations such as food and agriculture organization of the united nations (FAO) and world health organization (WHO), and have also been included in the “National Food Safety Standard for the Use of Food Additives” (GB2760) in China. Among stevia rebaudiana leaves, stevioside (St) and rebaudioside A (Reb A) are the most abundant, accounting for 5-10% (w/w) and 2-4% (w/w) of leaf dry weight, but the taste was poor (bitter and post-bitter), which affects its wide application. Rebaudioside D/M (Reb D/M) blend has the advantages of high sweetness (about 300 times that of sucrose), fast sweetening speed, long sweetness retention time, good thermal stability, low calorie and good taste (no peculiar taste and post-bitterness). It is the best sucrose substitute. However, the Reb D/M content only accounts for 0.4-0.5% (w/w) of the weight of dried stevia rebaudiana leaves, and the extraction amount of 1 ton of dried stevia leaves by traditional plant extraction method is about 2 kg. The process is cumbersome, polluted seriously and expensive, which seriously restricts the large-scale production of Reb D/M extracted from stevia leaves for commercial use.

Biological enzyme conversion method is the most technologically advantageous sustainable alternative strategy to achieve rebaudioside biosynthesis. The existing Reb D/M biological enzyme conversion method still has the following problems.
(1) High production cost. The expensive Reb D or Reb E are used as substrates (at levels well below 1%), and the expensive uridine diphosphate glucose (UDPG) is used as the donor substrate for glucosyl groups.
(2) Low substrate conversion and product accumulation. Whole cell catalysts using intracellularly expressing enzymes that hinder product synthesis due to resistance to substrate or product transport across membranes; and intracellular hydrolase (such as endogenous glycoside hydrolase spontaneous cell wall 2 (SCW2)) hydrolyzes the product to reduce the accumulation of the product.
(3) Low enzyme utilization rate. In co-catalysis employing a mixture of free, purified enzymes, several challenges are encountered: the pure enzymes are prepared through multiple, difficult steps and are easily inactivated. Furthermore, the free enzymes are difficult to be recovered and reused, and the downstream products are separated and purified with difficulty, resulting in high production costs. When traditional enzyme immobilization is used, additional immobilization steps are required. Immobilization materials are consumed, enzyme activity is reduced, and an irreversible decrease in the activity of the immobilized enzymes is observed over time.

Therefore, how to provide an efficient, sustainable, low-cost and high-strength green biomanufacturing technology of rebaudioside to break through the bottleneck of rebaudioside industrialization is an urgent problem for those skilled in the art to solve.

### SUMMARY

In view of this, the present invention has been provided.

An objective of the present invention is to provide double immobilized recombinant yeast engineering bacteria.

A second objective of the present invention is to provide a method for producing a double immobilized whole cell catalyst using the double immobilized recombinant yeast engineering bacteria.

A third objective of the present invention is to provide a method for producing rebaudioside using the double immobilized whole cell catalyst.

To achieve the above object, the present invention adopts the following technical solutions.

An example of the present invention provides a method for constructing double immobilized recombinant yeast engineering bacteria, including the steps of:
1) constructing a multi-enzyme complex immobilization module: linking a fragment 1 and a fragment 2 to a plasmid Episomal Saccharomyces cerevisiae-URA3 gene (PESC-URA) vector to obtain a recombinant PESC-URA vector; linking a fragment 3 to a plasmid RS424 (PRS424) vector to obtain a recombinant plasmid RS424 vector, wherein the fragment 1 is a surface display scaffold protein expression cassette included by a signal peptide, a yeast cell wall anchor protein, and an adhesion domain Cohesion; the fragment 2 is a UDPG in-situ regeneration system expression cassette included by a signal peptide, a yeast cell wall anchor protein, and sucrose synthase; and the fragment 3 is a multi-enzyme complex expression cassette included by promoters, a signal peptide, and an anchor domain Dockerin carrying enzyme subunits;
2) constructing a bacterial vector-free self-immobilization module: integrating a fragment 4 into a yeast genome, wherein the fragment 4 is a vector-free self-immobilization expression cassette included by a promoter and a flocculation gene; and
3) transformation: transferring the recombinant PESC-URA vector and the recombinant PRS424 vector into yeast cells to complete the construction of double immobilized recombinant yeast engineering bacteria.

In a preferred embodiment, in the fragment 1, the signal peptide is an α-factor signal peptide, the yeast cell wall anchor protein is an α-lectin Agα1, and the adhesion domain Cohesion scaffold protein is included by three cohesions: Cohesion 1 derived from Clostridium acetobutylicum, Cohesion 2 derived from Ruminiclostridium cellulolyticum and Cohesion 3derived from Clostridium cellulovorans; and the Agα1, Cohesion1, Cohesion2 and Cohesion3 are linked using a 3×G4S Linker, and nucleotide sequences are shown in SEQ ID NOs. 1, 9, 10 and 11; and
in the fragment 2, the signal peptide is the Aga2 signal peptide, the yeast cell wall anchor protein is α-agglutinin Aga2p, and the sucrose synthase is SUS01; Aga2p and SUS01 are linked using a 3×G4S Linker, and a gene sequence of this fusion is shown in SEQ ID NO.2; and an amino acid sequence of the sucrose synthase SUS01 is shown in SEQ ID NO.7, and a gene sequence thereof is shown in SEQ ID NO.13.

In a preferred embodiment, in the fragment 3, the promoters are phosphoglycerate kinase promoter (PGK), translation elongation factor 1 promoter (TEF1), and alcohol dehydrogenase 1 promoter (ADH1); the signal peptide is the α-factor signal peptide; the anchor domain Dockerin interacting proteins are Dockerin1 derived from Clostridium acetobutylicum (C. acetobutylicum) carrying uridine diphosphate glycosyltransferase 01 (UGT01), Dockerin2 derived from Ruminiclostridium cellulolyticum (R. cellulolyticum) carrying uridine diphosphate glycosyltransferase 02 (UGT02), and Dockerin3 derived from Clostridium cellulovorans (C. cellulovorans) carrying uridine diphosphate glycosyltransferase 01 (UGT01); and UGT01 and Dockerin1, UGT02 and Dockerin2, as well as UGT01 and Dockerin3 are linked using 3×G4S Linkers, and gene sequences are shown in SEQ ID NO.3, 14, and 15;
an amino acid sequence of the UGT01 is shown in SEQ ID NO.5; and
an amino acid sequence of the UGT02 is shown in SEQ ID NO.6.

In a preferred embodiment, in the fragment 4, the promoter is PGK, a flocculation gene is flocculation 1 short form (FLO1s), a nucleotide sequence of FLO1s is shown in SEQ ID NO.4, and an amino acid sequence of the flocculation protein FLO1s is shown in SEQ ID NO.8.

In a preferred embodiment, the yeast is any one of yeast hosts including Pichia pastoris, Saccharomyces cerevisiae and Kluyveromyces marxians; and

In a more preferred embodiment, the yeast is Saccharomyces cerevisiae AWY100.

A second aspect of the example of the present invention provides double immobilized recombinant yeast engineering bacteria constructed by the method described above.

A third aspect of the example of the present invention provides an application of the double immobilized recombinant yeast engineering bacteria in preparing a double immobilized recombinant yeast whole cell catalyst and synthesizing rebaudioside.

A fourth aspect of the example of the present invention provides a method for preparing the double immobilized recombinant yeast whole cell catalyst by the double immobilized recombinant yeast engineering bacteria, including the steps of:
inoculating activated dual-immobilized recombinant yeast engineering bacteria into a medium 1, followed by shaking culture at 20-40° C. and 150-300 rpm for 48-72 hours; and supplementing with a fresh medium 2, and conducting shaking culture at 20-40° C. and 150-300 rpm for 48-72 hours.

A fifth aspect of the example of the present invention provides a method for synthesizing RebD/M, including the steps of:

Adding St at a final concentration of 1-50 mmol/L or Reb A at 1-25 mmol/L, 10-500 mmol/L sucrose, 1-50 mmol/L uridine diphosphate (UDP), and 1-25 mmol/L calcium chloride (CaCl₂) to the double immobilized whole cell catalyst described in Claim 8; maintaining the pH stably in a range of 5.0-8.5 using 1-5 mol/L hydrochloric acid (HCl) and 10-50% ammonia water at 20-40° C.; catalyzing at 50-200 rpm for 1-5 hours and repeating the feeding operation 1-5 times; and achieving rapid separation of the whole cell catalyst from the reaction solution through bacterial self-sedimentation to terminate the reaction, and realizing the one-pot synthesis of Reb D/M; and
adding fresh reaction solution to directly proceed to the next round of rebaudioside biosynthesis after the self-sedimentation of the whole cell catalyst; and when the catalytic activity of the whole cell catalyst decreases to approximately 50-60% of the initial value, culturing for ⅓ to ½ of the culture time described in Claim 8 to complete the rejuvenation of the whole cell catalyst.

Preferably, the medium 1 is a synthetic defined-synthetic complete amino Acid (SD-SCAA)-leucine (Leu)-tryptophan (Trp)-uracil (Ura) medium, including 13.4 g/L of yeast nitrogen base (YNB), 0.6 g/L of dropout supplement without leucine/without tryptophan/without uracil (DO supplement-Leu/-Trp/-Ura), and 20 g/L glucose; and
the medium 2 is a high concentration medium including 67.0 g/L of YNB, 3 g/L of amino acid deletion mixture, 50 g/L of glucose, 100 g/L of galactose, and 20% of a volume of the medium 1 is supplemented.

As can be seen from the above technical solutions, compared with the related art, the technical effects achieved by the present invention are as follows.
1) In the present invention, the raw materials used are St and Reb A, which are abundant and cheap in stevia rebaudiana, and the sugar-based donor used is circularly supplied through the UDPG in-situ regeneration system, without adding expensive UDPG. Compared with the expensive substrate used in the related art, the present invention greatly reduces the production cost of RebD/M, has strong market competitiveness, and is conducive to occupying more market share.
2) In the present invention, a glycosyltransferase mutant UGT01 with high activity and high substrate selectivity is used, significantly altering the substrate selectivity of the enzyme, which is more inclined to convert St and Reb A to Reb D/M with little by-product Reb I. The relative enzyme activity of the glycosyltransferase mutant used is 5.4 times higher than that of the wild type, achieving efficient green synthesis of Reb D/M.
3) In the present invention, an ordered self-assembly multi-enzyme cascade catalytic system is constructed based on a substrate channel strategy of a fibrosome-like scaffold included by a nectin. Compared with the single-enzyme mixed co-catalysis or multi-enzyme disordered fixed catalysis mode in the prior art, the reaction intermediate of the present invention can quickly transfer from one active center to another active center, realizes efficient and orderly progress between multi-enzyme catalytic reactions, reduces the loss of competitive by-products and main products caused by disordered diffusion of the reaction intermediate, reduces the transportation time of the substrate, and improves the catalytic efficiency.
4) In the present invention, the enzyme immobilization technology coupled with surface-displayed multi-enzyme complexes and the flocculation carrier-free microbial self-immobilization technology are combined to construct a novel yeast whole cell catalyst with double immobilization of multi-enzyme complexes and microbial cells. Compared with the prior art, the advantages of the present invention are as follows: the transmembrane transport resistance of substrates and/or products, the diffusion or mass transfer resistance of traditional enzyme and/or microbial cell immobilization, the hydrolysis of products by intracellular hydrolases, and complex and tedious operating steps are avoided. At the same time, the whole cell catalyst can be rapidly separated from the reaction products by standing sedimentation, which can be reused many times and its catalytic activity is renewable after rejuvenation.

### BRIEF DESCRIPTION OF THE DRAWINGS

To explain the examples of the present invention or the technical solutions in the related art more clearly, a brief description will be given below with reference to the accompanying drawings which are used in the description of the examples or the prior art. Obviously, the drawings in the following description are only some examples of the present invention, and other drawings can be obtained according to these drawings supplies without creative work for those ordinary skilled in the art.

FIG. 1A is a structural diagram of a recombinant vector 1;

FIG. 1B is a structural diagram of recombinant vector 2;

FIG. 1C is a structural diagram of an integrated fragment;

FIG. 2 shows sodium dodecyl sulfate-polyacrylamide gel electrophoresis (SDS-PAGE) of glycosyltransferases UGT01, UGT02, and sucrose synthase SUS01;

FIG. 3A shows a substrate interaction relationship of UGT76G1;

FIG. 3B shows a comparison of catalytic activities between UGT76G1 and UGT01;

FIG. 4 shows a comparison between the synthesis of rebaudiosides by free enzyme co-catalysis and multi-enzyme cascade catalysis;

FIG. 5 shows a comparison of relative activities of free, embedded and flocculated self-immobilized whole cell catalysts;

FIG. 6 shows the effect of one-time feeding and batch feeding methods on the synthesis of rebaudioside;

FIG. 7A is a schematic diagram of self-sedimentation of bacteria; and

FIG. 7B shows relative enzyme activities of rejuvenated strains.

### DETAILED DESCRIPTION

Technical solutions in the examples of the present invention will be described clearly and completely in the following with reference to the accompanying drawings in the examples of the present invention. Obviously, all the described examples are only some, rather than all examples of the present invention. Based on the examples in the present invention, all other examples obtained by those ordinary skilled in the art without creative efforts belong to the scope of protection of the present invention.

### Example 1 Construction of Double Immobilized Recombinant Yeast Engineered Bacteria

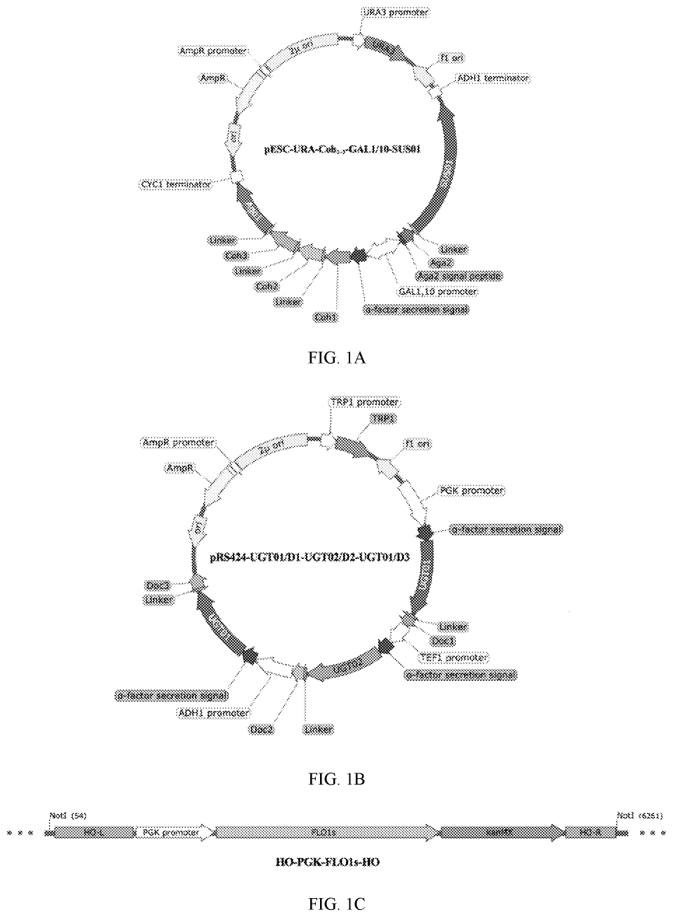

A fragment 1 and a fragment 2 were obtained by whole gene synthesis and ligated to pESC-URA vector, and a constructed recombinant vector 1 is shown in FIG. 1A. A fragment 3 was obtained by whole gene synthesis and ligated to pRS424 vector, and a constructed recombinant vector 2 is shown in FIG. 1B. A fragment 4 carrying upper and lower homology arms at two ends was obtained by whole gene synthesis, and a constructed integrated fragment is shown in FIG. 1C. After a FLO1s fragment including the flocculation gene was integrated into a genome of Saccharomyces cerevisiae AWY100, the recombinant vector 1 and the recombinant vector 2 were introduced by electric shock transformation method to construct double immobilized recombinant yeast engineering bacteria AWY100-pESC-C₁₋₃-GAL1/10-SUS01-pRS424-UGT01/D₁-UGT02/D₂-UGT01/D₃-FLO1s, abbreviated as “AWY100-FSUD”
1) A fragment including the flocculation gene FLO1s (as shown in SEQ ID NO.4) was integrated into the genome of Saccharomyces cerevisiae AWY100. Subsequently, the recombinant pESC-URA vector and recombinant pRS424 vector were introduced via electroporation, and the dual-immobilized recombinant yeast engineering strain AWY100-pESC-C₁₋₃-GAL1/10-SUS01-pRS424-UGT01/D₁-UGT02/D₂-UGT01/D₃-FLO1s was constructed.

The fragment 1 is a surface display scaffold protein expression cassette included by a signal peptide, a yeast cell wall anchor protein, and an adhesion domain Cohesion; the fragment 2 is a UDPG in-situ regeneration system expression cassette includes by a signal peptide, a yeast cell wall anchor protein, and sucrose synthase; and the fragment 3 is a multi-enzyme complex expression cassette included by promoters, a signal peptide, and an anchor domain Dockerin carrying enzyme subunits.
2) A bacterial vector-free self-immobilization module was constructed: a fragment 4 was integrated into a yeast genome, wherein the fragment 4 was a vector-free self-immobilization expression cassette included by a promoter and a flocculation gene.
3) transformation: the recombinant PESC-URA vector and the recombinant PRS424 vector were transferred into yeast cells to complete the construction of double immobilized recombinant yeast engineering bacteria.

In the fragment 1, the signal peptide is an α-factor signal peptide, the yeast cell wall anchor protein is an α-lectin Agα1, and the adhesion domain Cohesion scaffold protein is included by three cohesions: Cohesion 1 derived from Clostridium acetobutylicum, Cohesion 2 derived from Ruminiclostridium cellulolyticum and Cohesion 3 derived from Clostridium cellulovorans; and the Agα1, Cohesion1, Cohesion2 and Cohesion3 are linked using a 3×G4S Linker, and nucleotide sequences are shown in SEQ ID NOs.1, 9, 10 and 11.

In the fragment 2, the signal peptide is the Aga2 signal peptide, the yeast cell wall anchor protein is α-agglutinin Aga2p, and the sucrose synthase is SUS01; Aga2p and SUS01 are linked using a 3×G4S Linker, and a gene sequence of this fusion is shown in SEQ ID NO.2; and an amino acid sequence of the sucrose synthase SUS01 is shown in SEQ ID NO.7, and a gene sequence thereof is shown in SEQ ID NO.13.

In the fragment 3, the promoters are PGK, TEF1, and ADH1; the signal peptide is the α-factor signal peptide; the anchor domain Dockerin interacting proteins are Dockerin1 derived from C. acetobutylicum carrying UGT01, Dockerin2 derived from R. cellulolyticum carrying UGT02, and Dockerin3 derived from C. cellulovorans carrying UGT01; and UGT01 and Dockerin1, UGT02 and Dockerin2, as well as UGT01 and Dockerin3 are linked using 3×G4S Linkers, and gene sequences are shown in SEQ ID NO.3, 14, and 15.

An amino acid sequence of the UGT01 is shown in SEQ ID NO.5.

An amino acid sequence of the UGT02 is shown in SEQ ID NO.6.

In the fragment 4, the promoter is PGK, a flocculation gene is FLO1s, a nucleotide sequence of FLO1s is shown in SEQ ID NO.4, and an amino acid sequence of the flocculation protein FLO1s is shown in SEQ ID NO.8.

### Example 2 Protein Expression of Biological Enzymes UGT01, UGT02 and SUS01

Protein expression was characterized by SDS-PAGE electrophoresis. Protein bands and protein Marker reference band positions were compared.

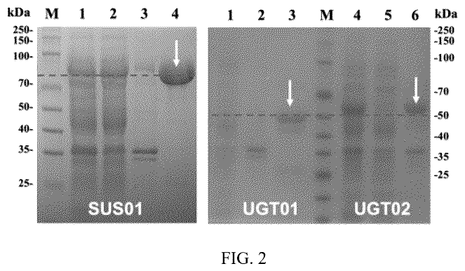

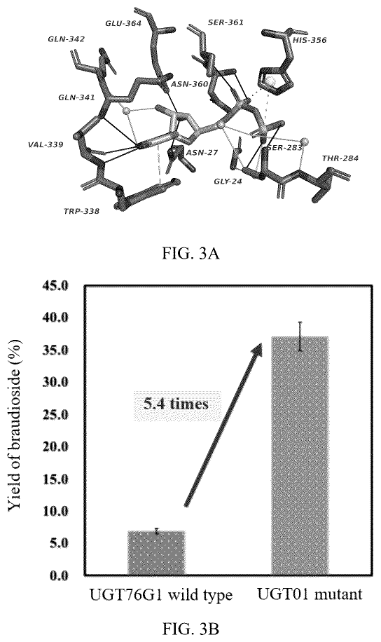

According to the technical solution provided by the present invention, heterologous soluble expression of sucrose synthase SUS01, glycosyltransferase UGT01 and UGT02 in a yeast host was successfully achieved. Characterized by SDS-PAGE protein electrophoresis, the molecular weight of SUS01 is about 85 kDa, and the molecular weights of UGT01 and UGT02 are about 50 kDa and 55 kDa, which are basically consistent with the theoretical molecular weight. The results are shown in FIG. 2.

### Example 3 Comparison of Glycosyltransferase UGT01 Mutant With UGT76G1 Wild Type

Glycosyltransferase UGT76G1 is the most critical rate-limiting enzyme in the enzymatic biosynthesis of rebaudioside. To improve the yield of rebaudioside, wild-type UGT76G1 was subjected to directed evolution and multiple rounds of rational design and modification. Based on the previously reported substrate interaction relationship of the crystal structure of UGT76G1, the uridine ring of the substrate forms T-stacking with W338; forms a series of hydrogen bond positioning networks with D27, S283, V339, N360, S361, and E364 to assist in the positioning of the substrate UDP; and forms a salt bridge with H356. Based on the above functional relationships, a series of mutant libraries were designed using rational mutation methods. For W338, it was replaced with F and H, which produce similar interactions, to fine-tune the π-π interactions; the six amino acids D27, S283, V339, N360, S361, and E364 were subjected to iterative saturation mutagenesis using the NNK codon method to adjust the substrate positioning mode and improve catalytic activity; while H356, verified by sequence alignment, is highly conserved at this site and thus was not mutated. By combining and superimposing the mutants from the rational design strategy and the random mutations of error-prone polymerase chain reaction (error-prone PCR), the glycosyltransferase mutant UGT01 was obtained.

The enzymes were added to a final concentration system containing 1-50 mmol/L St or 1-25 mmol/L Reb A, 10-500 mmol/L sucrose, 1-50 mmol/L UDP, and 1-25 mmol/L CaCl₂. The reaction was carried out at 20-40° C. with a stirring speed of 50-200 rpm for 1-5 hours. The pH was maintained stable within the range of 5.0-8.5 using 1-5 mol/L HC1 and 10-50% ammonia water. The concentration of rebaudiosides was detected by high performance liquid chromatography (HPLC).

HPLC: liquid chromatography method: C18 column (length: 250 mm; inner diameter: 4.6 mm; and particle size: 5 μm). The mobile phase was a 68:32 (v/v) mixture of 1.38 g/L sodium phosphate buffer and acetonitrile (pH 2.6), with a flow rate of 1.0 mL/min, a column temperature of 40° C., a detection wavelength of 210 nm, and an injection volume of 10 μL.

The yield of rebaudioside synthesized by mutant UGT01 was 37.1% within 30 min, while the yield of wild-type UGT76G1 was only 6.9%, and the catalytic activity of UGT01 was 5.4-fold higher than that of wild-type. At the same time, UGT01 changed the substrate selectivity, reduced the synthesis of by-product Reb I, and made the catalytic reaction process proceed towards the synthesis direction of the main product Reb D/M.

### Example 4 Comparison of Free Enzyme Co-catalysis and Multi-enzyme Cascade Catalysis

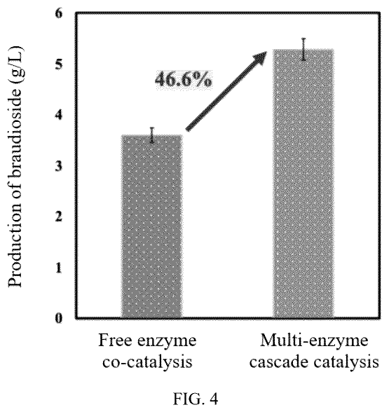

According to the technical solutions provided by the present invention, the following recombinant yeast engineering bacteria were cultured. Other culture conditions were kept consistent. Recombinant bacteria without the addition of inducer could not express surface-displayed scaffold proteins to self-assemble into multi-enzyme cascade complexes, so this group was designated as the free enzyme co-catalysis experimental group; while recombinant bacteria with the addition of inducer were designated as the multi-enzyme cascade catalysis experimental group. UDPG was added directly during the reaction without enabling the UDPG in situ regeneration system to reduce interference factors, and other reaction conditions were described above. The results are shown in FIG. 4, and the rebaudioside yield catalyzed by multi-enzyme cascade is 46.6% higher than that catalyzed by free enzyme co-catalysis.

### Example 5 Comparison of Free, Embedded and Flocculated Self-immobilized Whole Cell Catalysts

According to the technical solutions provided by the present invention, the following recombinant yeast engineering bacteria were cultured. The recombinant pESC-URA vector and recombinant pRS424 vector were introduced into Saccharomyces cerevisiae host AWY100 without integration of flocculation gene, and the free recombinant bacterium displaying multi-enzyme cascade complex on the surface was constructed, which was named A_{free}. 1.5% sodium alginate was used to make embedded immobilized pellets, named A_{embedding}, and the present invention constructed high-performance double-immobilized recombinant bacteria A_{flocculation} in Example 1.

Activity detection conditions: to the dual-immobilized whole-cell catalyst, substrates were added to reach final concentrations of 1-50 mmol/L St or 1-25 mmol/L Reb A, 10-500 mmol/L sucrose, 1-50 mmol/L UDP, and 1-25 mmol/L CaCl₂. The reaction was conducted at 20-40° C., with the pH maintained stable within the range of 5.0-8.5 using 1-5 mol/L HCl and 10-50% ammonia water, and catalyzed at 50-200 rpm for 1-5 hours. The feeding operation was repeated 1-5 times. Finally, the whole cell catalyst was rapidly separated from the reaction solution by microbial self-sedimentation to terminate the reaction, and the one-pot synthesis of rebaudiosides Reb D/M was achieved.

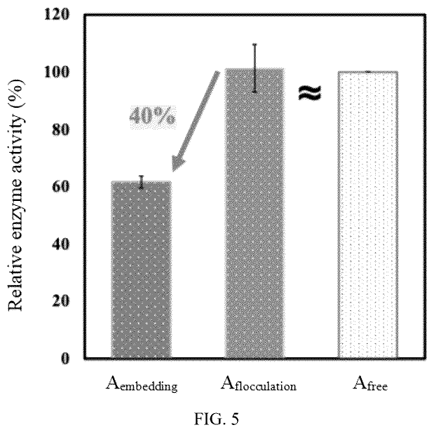

The results are shown in FIG. 5, the relative activities of A_{flocculation} and A_{free} are basically similar, indicating that the flocculation self-immobilization method has no negative impact on the catalytic functional domain, and there is almost no mass transfer resistance. On the contrary, flocculation agglomeration increased bacterial density (population effect) and endowed strains with better tolerance to environmental stress. However, A_{embedding} is affected by mass transfer resistance, and the reaction substrate/intermediate cannot quickly enter the enzyme active center through the entrapment material, resulting in a significantly slower catalytic speed.

### Example 6 Effect of One-time Feeding and Batch Feeding on the Synthesis of Rebaudioside

According to the technical proposal provided by the present invention, the high-performance double immobilized recombinant yeast engineering bacteria constructed by the present invention were cultured. In this example, the effects of different substrate addition methods on rebaudioside synthesis were studied by one-time feeding and batch feeding methods.

One-time feeding: 30 mmol/L St substrate was charged into the reaction solution at one time.

Batch feeding: 10 mmol/L St was put into the reaction solution as the initial substrate, and fed every 2.5 h for a total of 2 feedings.

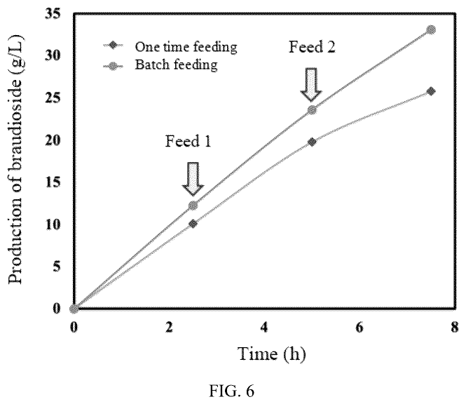

The results are shown in FIG. 6. Compared with one-time feeding, batch feeding can greatly increase the yield of rebaudioside. 10 mmol/L St is the initial substrate, and the feeding is fed every 2.5 h. After 2 feedings, the yield of Reb D/M is 33.1 g/L.

### Example 7 Reuse and Rejuvenation of Double Immobilized Recombinant Yeast Whole Cell Catalyst

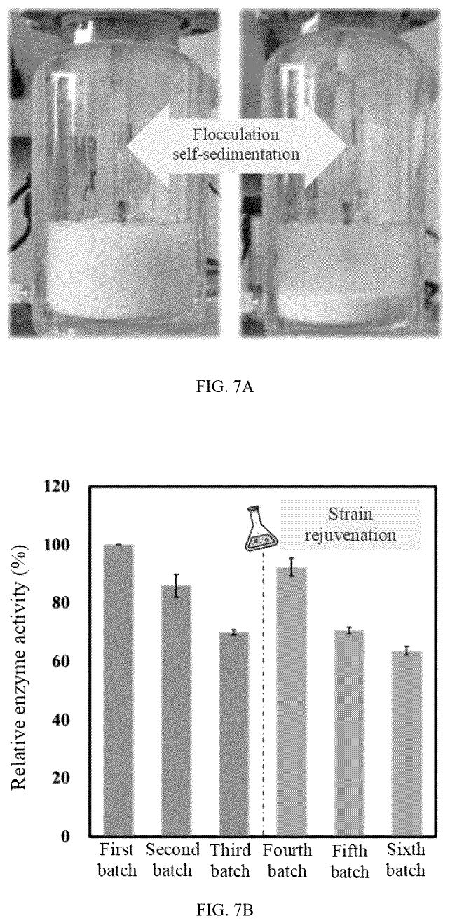

The double immobilized recombinant yeast engineering bacteria synthesize rebaudioside by batch feeding as described in Example 6. Every time a round of catalytic reaction is completed, the reaction solution is quickly separated by self-sedimentation of the bacteria, FIG. 7A, and fresh reaction solution is supplemented to enter the next cycle. When the relative enzymatic activity of the whole-cell catalyst was detected to be less than 60% of the initial value, the cycle was stopped and the strain rejuvenation stage was initiated. According to the technical solutions provided by the present invention, only one third of the initial culture time of the bacterial cells can be used to complete the strain rejuvenation. As shown in FIG. 7B, a multi-enzyme complex double immobilized recombinant yeast whole cell catalyst created by the present invention can maintain good catalytic stability in six cycles, showing the potential as an industrial strain for synthesizing natural sweetener Reb D/M with high efficiency and low cost, and has important practical significance and economic value for promoting China's international competitiveness in the field of food processing.

Each example in this specification is described in a progressive manner, and each example focuses on the differences from other examples. The same and similar parts of each example can be referred to each other.

The foregoing description of the disclosed examples enables those skilled in the art to implement or use the present invention. Various modifications to these examples will be apparent to those skilled in the art, and the general principles defined herein may be implemented in other examples without departing from the spirit or scope of the present invention. Thus, the present invention is not intended to be limited to the embodiments shown herein but is to be accorded the widest scope consistent with the principles and novel features disclosed herein.

What is claimed is:

1. A construction method of double immobilized recombinant yeast engineering bacteria, comprising the steps of:
1) constructing a multi-enzyme complex immobilization module: linking a fragment 1 and a fragment 2 to a plasmid Episomal Saccharomyces cerevisiae-URA3 gene (PESC-URA) vector to obtain a recombinant PESC-URA vector; linking a fragment 3 to a plasmid RS424 (PRS424) vector to obtain a recombinant plasmid RS424 vector, wherein the fragment 1 is a surface display scaffold protein expression cassette comprised by a signal peptide, a yeast cell wall anchor protein, and an adhesion domain Cohesion; the fragment 2 is a uridine piphosphate glucose (UDPG) in-situ regeneration system expression cassette comprised by a signal peptide, a yeast cell wall anchor protein, and sucrose synthase; and the fragment 3 is a multi-enzyme complex expression cassette comprised by promoters, a signal peptide, and an anchor domain Dockerin carrying enzyme subunits;
2) constructing a bacterial vector-free self-immobilization module: integrating a fragment 4 into a yeast genome, wherein the fragment 4 is a vector-free self-immobilization expression cassette comprised by a promoter and a flocculation gene; and
3) transformation: transferring the recombinant PESC-URA vector and the recombinant PRS424 vector into yeast cells to complete the construction of double immobilized recombinant yeast engineering bacteria.

2. The method according to claim 1, wherein in the fragment 1, the signal peptide is an α-factor signal peptide, the yeast cell wall anchor protein is an α-lectin Agα1, and the adhesion domain Cohesion scaffold protein is comprised by three cohesions: Cohesion 1 derived from Clostridium acetobutylicum, Cohesion 2 derived from Ruminiclostridium cellulolyticum and Cohesion 3 derived from Clostridium cellulovorans; and the Agα1, Cohesion1, Cohesion2 and Cohesion3 are linked using a 3×G4S Linker, and nucleotide sequences are shown in SEQ ID NOs.1, 9, 10 and 11; and
in the fragment 2, the signal peptide is Aga2 signal peptide, the yeast cell wall anchor protein is α-lectin Aga2p, and the sucrose synthase is SUS01; Aga2p and SUS01 are linked using a 3×G4S Linker, and a gene sequence of Aga2p is shown in SEQ ID NO.2; and an amino acid sequence of the sucrose synthase SUS01 is shown in SEQ ID NO.7, and a gene sequence thereof is shown in SEQ ID NO.13.

3. The method according to claim 1, wherein in the fragment 3, the promoters are phosphoglycerate kinase promoter (PGK), translation elongation factor 1 promoter (TEF1), and alcohol dehydrogenase 1 promoter (ADH1); the signal peptide is the α-factor signal peptide; the anchor domain Dockerin interacting proteins are Dockerin1 derived from Clostridium acetobutylicum (C. acetobutylicum) carrying uridine diphosphate glycosyltransferase 01 (UGT01), Dockerin2 derived from Ruminiclostridium cellulolyticum (R. cellulolyticum) carrying uridine diphosphate glycosyltransferase 02 (UGT02), and Dockerin3 derived from Clostridium cellulovorans (C. cellulovorans) carrying uridine diphosphate glycosyltransferase 01 (UGT01), and gene sequences are shown in SEQ ID NO.3, 14, and 15; and UGT01 and Dockerin1, UGT02 and Dockerin2, as well as UGT01 and Dockerin3 are linked using 3×G4S Linkers, and an amino acid sequence of the UGT01 is shown in SEQ ID NO.5; and
an amino acid sequence of the UGT02 is set forth in SEQ ID NO.6.

4. The method according to claim 1, wherein in the fragment 4, the promoter is PGK, a flocculation gene is flocculation 1 short form (FLO1s), a nucleotide sequence of FLO1s is shown in SEQ ID NO.4, and an amino acid sequence of the flocculation protein FLO1s is shown in SEQ ID NO.8.

5. The method according to claim 1, wherein the yeast is any one of yeast hosts comprising Pichia pastoris, Saccharomyces cerevisiae and Kluyveromyces marxians; and
preferably, the yeast is Saccharomyces cerevisiae AWY100.

6. A double immobilized recombinant yeast engineering bacterium constructed by the method according to claim 1.

7. An application of the double immobilized recombinant yeast engineering bacteria according to claim 6 in preparing a double immobilized recombinant yeast whole cell catalyst and synthesizing rebaudioside.

8. A method for preparing the double immobilized recombinant yeast whole cell catalyst using the double immobilized recombinant yeast engineering bacteria according to claim 6, comprising the steps of:
inoculating activated dual-immobilized recombinant yeast engineering bacteria into a medium 1, followed by shaking culture at 20-40° C. and 150-300 rpm for 48-72 hours; and supplementing with a fresh medium 2, and conducting shaking culture at 20-40° C. and 150-300 rpm for 48-72 hours.

9. A method for synthesizing rebaudioside D/M (RebD/M), comprising the steps of:
adding stevioside (St) at a final concentration of 1-50 mmol/L or rebaudioside A (Reb A) at 1-25 mmol/L, 10-500 mmol/L sucrose, 1-50 mmol/L uridine diphosphate (UDP), and 1-25 mmol/L calcium chloride (CaCl₂) to the double immobilized whole cell catalyst described in claim 8; maintaining the pH stably in a range of 5.0-8.5 using 1-5 mol/L hydrochloric acid (HCl) and 10-50% ammonia water at 20-40° C.; catalyzing at 50-200 rpm for 1-5 hours and repeating the feeding operation 1-5 times; and achieving rapid separation of the whole cell catalyst from the reaction solution through bacterial self-sedimentation to terminate the reaction, and realizing the one-pot synthesis of Reb D/M; and
adding fresh reaction solution to directly proceed to the next round of rebaudioside biosynthesis after the self-sedimentation of the whole cell catalyst; and when the catalytic activity of the whole cell catalyst decreases to approximately 50-60% of the initial value, culturing for 24-36 hours to complete the rejuvenation of the whole cell catalyst.

10. The method according to claim 9, wherein the medium 1 is a synthetic defined-synthetic complete amino Acid (SD-SCAA)-leucine (Leu)-tryptophan (Trp)-uracil (Ura) medium, comprising 13.4 g/L of yeast nitrogen base (YNB), 0.6 g/L of dropout supplement without leucine/without tryptophan/without uracil (DO supplement-Leu/-Trp/-Ura), and 20 g/L glucose; and
the medium 2 is a high concentration medium comprising 67.0 g/L of YNB, 3 g/L of amino acid deletion mixture, 50 g/L of glucose, 100 g/L of galactose, and 20% of a volume of the medium 1 is supplemented.
